(19) United States
(12) Patent Application Publication
(10) Pub. No.: US 2026/0041545 A1
(43) Pub. Date: Feb. 12, 2026

(54) Ligament/Tendon Suture with Paired Anchor System and Syndesmosis Repair Jig

(71) Applicant: Tyber Medical LLC, Bethlehem, PA (US)

(72) Inventor: Robert Flavin, Enniskerry (IE)

(21) Appl. No.: 19/102,172

(22) PCT Filed: Aug. 15, 2023
(86) PCT No.: PCT/US23/30186

§ 371 Date: Feb. 7, 2025

Related U.S. Application Data
(60) Provisional application No. 63/402,961, filed on Sep. 1, 2022.
(60) Provisional application No. 63/402,971, filed on Sep. 1, 2022.
(60) Provisional application No. 63/428,989, filed on Nov. 30, 2022.

(51) Int. Cl. A61F 2/08; A61B 17/00; A61B 17/86; A61B 17/88

(52) U.S. Cl. CPC A61F 2/0811; A61B 17/8645; A61B 17/888; A61B 2017/00004; A61F 2002/0817; A61F 2002/0852; A61F 2002/0888

(57) ABSTRACT

A suture and anchor delivery system includes a rotation suture, a tendon/ligament suture, and an anchor. The anchor includes a generally cylindrical body having a length, a width, a proximal end, and a distal end terminating in a bullet tip. The bullet tip has a first pair of diametrically opposing through-openings, wherein the rotation suture is inserted therethrough. The body further has a second pair of diametrically opposed through openings, wherein the tendon/ligament suture is inserted therethrough. An anchor delivery device includes a hollow tubular body configured to allow the anchor to be inserted therein. The tubular body has an open distal tip from which the anchor is discharged and a plunger head slidingly disposed in the tubular body. The plunger head has a distal driver sized to engage the open channel and drive the anchor distally into a bone.

### BACKGROUND OF THE INVENTION

### Field of the Invention

The invention relates to a system for repairing a damaged ligament/tendon by providing a suture to effect the repair.

### Description of the Related Art

Sutures for ligament and tendon repair are typically non-resorbable, which can leave palpable knots or increase the risk of infection, or resorbable, which means that the suture tends to dissolve over a confined period of time, requiring the ligament or tendon to fully heal before the suture fully resorbs.

It would be beneficial to provide a suture that resorbs at different rates to allow the ligament or tendon time to heal in stages rather than all at once.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a suture and anchor delivery system. The system includes a rotation suture, a tendon/ligament suture, and an anchor. The anchor includes a generally cylindrical body having a length, a width, a proximal end, and a distal end terminating in a bullet tip. The bullet tip has a first pair of diametrically opposing through-openings, wherein the rotation suture is inserted therethrough. The body further has a second pair of diametrically opposed through openings, wherein the tendon/ligament suture is inserted therethrough. An anchor delivery device includes a hollow tubular body configured to allow the anchor to be inserted therein. The tubular body has an open distal tip from which the anchor is discharged and a plunger head slidingly disposed in the tubular body. The plunger head has a distal driver sized to engage the open channel and drive the anchor distally into a bone.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of a bone anchor system with a suture according to an exemplary embodiment of the invention;

FIG. 2 is a perspective view of the bone anchor system without the suture of FIG. 1;

FIG. 3 is a perspective view, in section, of the bone anchor system of FIG. 1;

FIG. 4 is a perspective view of the bone anchor of the system of FIG. 1;

FIG. 5 is a perspective view, in section, of the bone anchor of FIG. 4 taken along lines 5-5 of FIG. 4;

FIG. 6 is a perspective view, in section, of the bone anchor of FIG. 4 taken along lines 6-6 of FIG. 4

FIG. 7 is a perspective view of a plug of the anchor system of FIG. 1;

FIG. 8 is an alternative embodiment of a plug of the system of FIG. 1;

FIG. 9 is a side perspective view of a bone anchor system according to an alternative embodiment of the present invention;

FIG. 10 is a top perspective view of the bone anchor system of FIG. 9;

FIG. 11 is a side perspective view of a bone anchor system according to yet another alternative embodiment of the present invention;

FIG. 12 is a front elevational view of a rotator cuff for repair by an exemplary embodiment of the present invention;

FIG. 13 is a side elevational view of an anchor system used to repair the rotator cuff of FIG. 12;

FIG. 14 is a perspective view of an anchor used in the anchor system of FIG. 13;

FIG. 15 is a side elevational view of the anchor of FIG. 14;

FIG. 16 is a top plan view of the anchor of FIG. 14;

FIG. 17 is a front elevational view of the anchor of FIG. 14;

FIG. 18 is a side elevational view of a delivery device delivering the anchor of FIG. 14 into a shoulder joint;

FIG. 19 is a side elevational view, in sectopn. of the anchor being inserted into the shoulder joint of FIG. 18;

FIG. 20 is a side elevational view of the use of the delivery device being operated to rotate the anchor inside the shoulder joint;

FIG. 21 is a side elevational view, in section, of the anchor having been rotated in the shoulder joint;

FIG. 22 is a perspective view of a syndesmosis repair jig according to an exemplary embodiment of the present invention;

FIG. 23 is a side elevational view of the jig of FIG. 22;

FIG. 24 is a bottom plan view of the jig of FIG. 22;

FIG. 25 is an end elevational view of the jig of FIG. 22;

FIG. 26 is a perspective view of the jig of FIG. 22 attached to a tibia and fibula and used to repair a syndesmosis ligament;

FIG. 27 is a side elevational view of the jig attached to the tibia and fibula of FIG. 26;

FIG. 28 is a bottom plan view of the jig attached to the tibia and fibula of FIG. 26;

FIG. 29 is an end elevational view of the jig attached to the tibia and fibula of FIG. 26; and

FIG. 30 is a schematic view showing a suture securing the tibia and fibula to each other.

### DETAILED DESCRIPTION

U.S. Provisional Patent Application Ser. No. 63/402,962, entitled “Screw Capture System for Calcaneal Fracture”, filed on Sep. 1, 2022 and invented by this inventor (Attorney Docket No. TYB-057); U.S. Provisional Patent Application Ser. No. 63/402,965, entitled “Screw Blade Capture System”, filed on Sep. 1, 2022 and invented by this inventor (Attorney Docket No. TYB-058); U.S. Provisional Patent Application Ser. No. 63/402,967, entitled “Repair Plate System from Inside/Out”, filed on Sep. 1, 2022 and invented by this inventor (Attorney Docket No. TYB-059); U.S. Provisional Patent Application Ser. No. 63/402,964, entitled “Talar Repair Jig”, filed on Sep. 1, 2022 and invented by this inventor (Attorney Docket No. TYB-060); U.S. Provisional Patent Application Ser. No. 63/402,971, entitled “Syndesmosis Repair Jig System and Method of Use”, filed on Sep. 1, 2022 and invented by this inventor (Attorney Docket No. TYB-061); U.S. Provisional Patent Application Ser. No. 63/402,969, entitled “Distal Tibial Osteotomy System”, filed on Sep. 1, 2022 and invented by this inventor (Attorney Docket No. TYB-062); U.S. Provisional Patent Application Ser. No. 63/402,963, entitled “External Fixation System”, filed on Sep. 1, 2022 and invented by this inventor (Attorney Docket No. TYB-063); and U.S. Provisional Patent Application Ser. No. 63/402,970, entitled “Achilles Tendon Repair Jig”, filed on Sep. 1, 2022 and invented by this inventor (Attorney Docket No. TYB-064), are all incorporated herein by reference in their entireties.

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately”preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

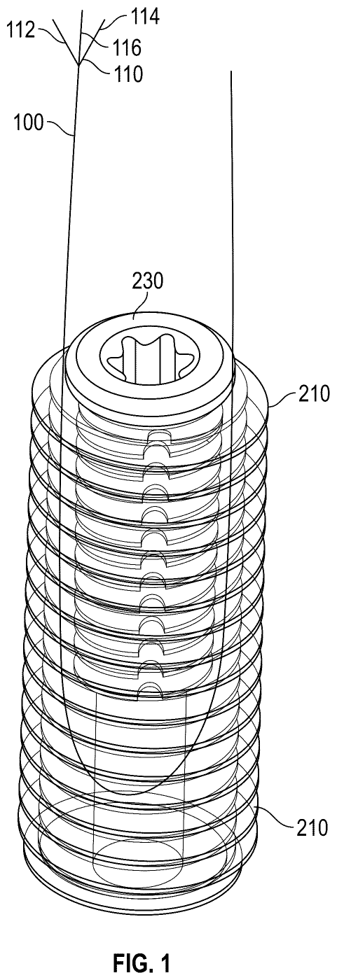

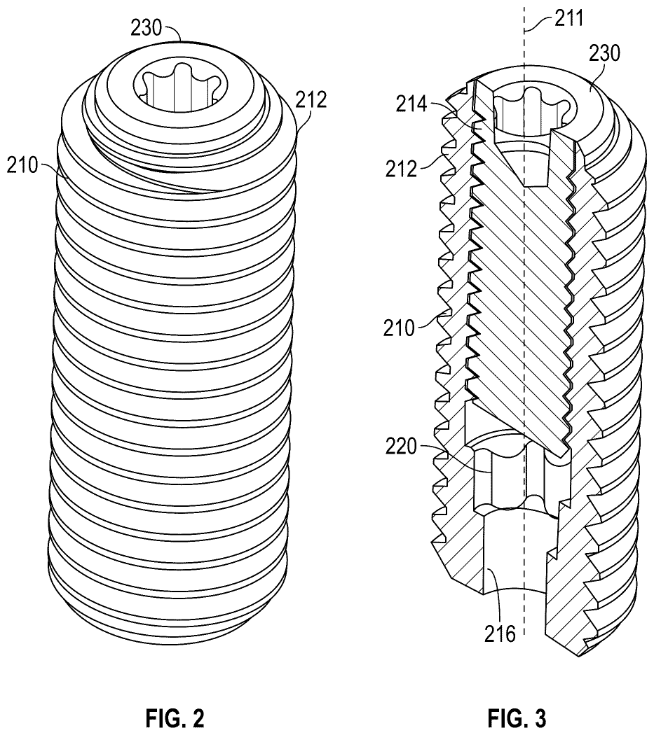

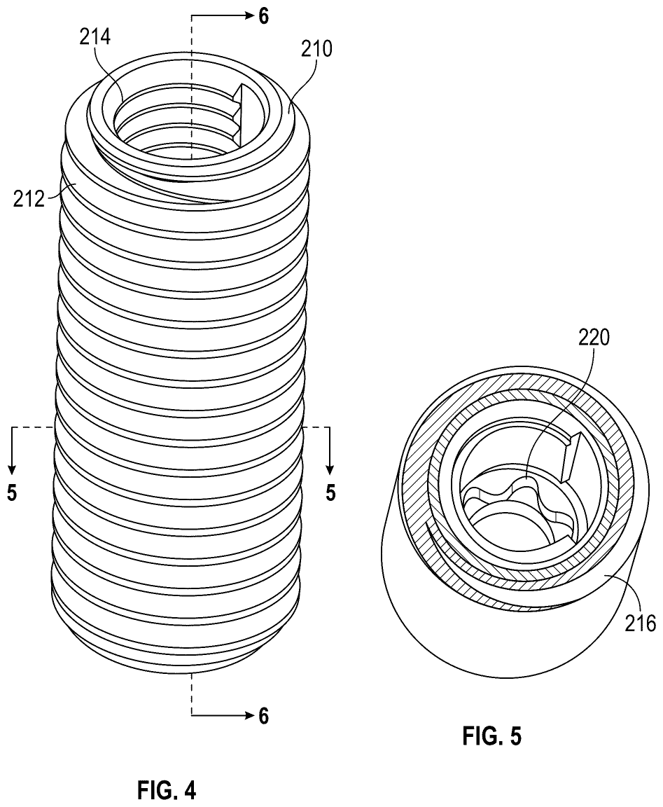

Referring to FIG. 1, a partially resorbable suture 100 (“suture 100”) according to a first exemplary embodiment of the present invention is shown. While suture 100 is discussed herein as used to repair Achilles tendon injuries, those skilled in the art will recognize that suture 100 can be used for other types of soft tissue reconstruction or biologic repair. In an exemplary embodiment, suture 100 n be radiopaque.

Suture 100 includes a plurality of strands 110 that are woven together to make a single thread. Suture 100 is a multifilament hybrid absorbable/non-absorbable braided surgical suture that combines at least two separate monofilament materials, at least one of which is absorbable and one of which is not, via a braided construction. The hybrid surgical suture 100 may be formed by braiding yarns of non-absorbable high tenacity fibers and absorbable monofilaments. A first set 112 of the plurality of strands 110 can be constructed from polyglycolic acid, polydioxanone, other biodegradable material, or combinations thereof that biodegrades over a first period of time, such as, for example, about four weeks after implantation into a patient. As the first set 112 biodegrades, the tendon strengthens to compensate for the loss of strength in suture 100.

A second set 114 of the plurality of strands 110 can be constructed from polyglycolic acid, polydioxanone, other biodegradable material, or combinations thereof that biodegrades over a second period of time, longer than the first period of time. An exemplary duration of time can be about 6-9 months after implantation into the patient. As the second set 114 degrades, the tendon further strengthens to further compensate for the loss of strength in suture 100.

While two sets 112, 114 of biodegradable strands are disclosed, those skilled in the art will recognize that more or less than two sets of biodegradable strands can be used.

Remaining set 116 of strands 110 can be constructed from a material that does not biodegrade over time, such as nylon, silk, polyester, polyethylene or polypropylene and remains to assist the tendon repair itself as the tendon heals. The combination of permanent and absorbable properties of the hybrid surgical suture 100 allow the suture 100 itself to downsize over time; this allows the suture 100 to be used for procedures that require a level of permanent fixation, while allowing the regrowth of biologic material as the absorbable materials dissipate. In an exemplary embodiment, the remaining set 116 comprises about 20% of the suture 100 by weight.

As first set 112 and second set 114 biodegrade, suture 10 reduces in bulk size over time, consequently reducing the size of any knot that is tied in suture 100. In an exemplary embodiment, after second set 114 degrades, suture 100 is reduced in size by to less than 30% of its original bulk size.

Optionally, first set 112, second set 114, and third set 116 of strands can be individually coated with a bacteriostatic material. Alternatively, each individual strand that ultimately comprises suture 100 can be coated with the bacteriostatic material. Still alternatively, the total suture 100 can be coated with the bacteriostatic material.

Suture 100 can be used in an anchor system 200, shown in FIGS. 1—Anchor system 200 can be installed in a posterior drill hole in a fibula (not shown), with suture 100 extending from anchor system 200.

Anchor system 200 includes a sleeve 210 that has a generally hollow body extending along a longitudinal axis 211. Sleeve 210 can be constructed form titanium or other biocompatible material. Sleeve 210 includes an external thread 212 that secures sleeve 210 to the fibula. Sleeve also includes an internal thread 214 that mates with a corresponding plug 230.

Referring to FIG. 5, a distal end 216 of sleeve features a drive socket 220 formed therein. Drive socket 220 is sized to allow a driver, such as a Torx® driver (not shown), to be inserted through sleeve 210 engage drive socket 220 and rotate sleeve 210 for insertion into the fibula.

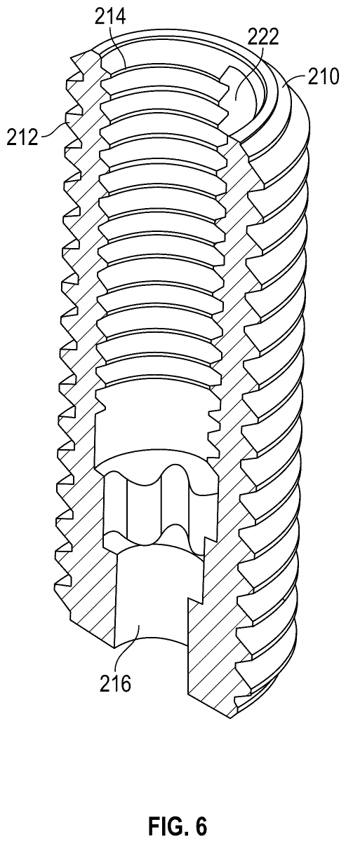

Referring to FIG. 6, internal thread 214 can be broken to form a longitudinal channel 222 parallel to longitudinal axis 211. Although not shown, a diametrically opposite channel can also be formed. This channel and channel 222 form a passage for suture 100 to extend through sleeve 210 without interfering with plug 230.

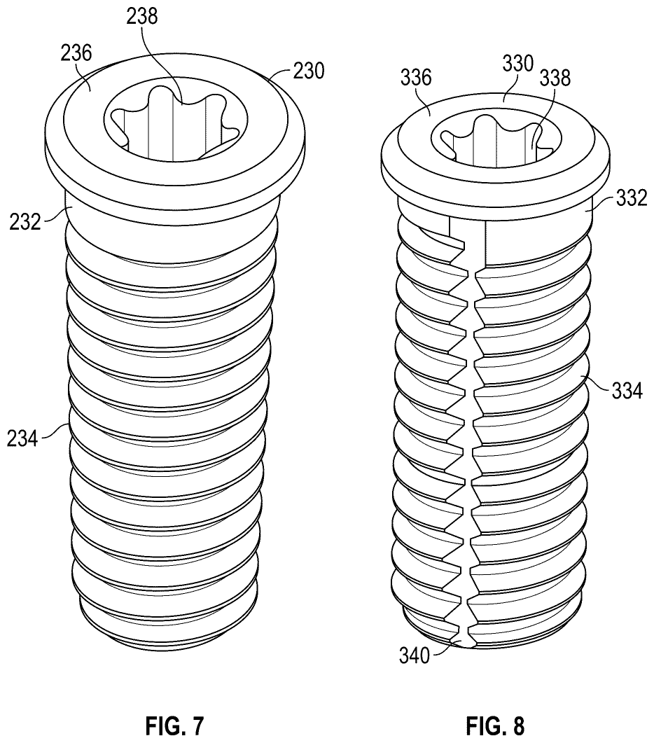

Referring to FIG. 7, plug 230 has an elongate tubular body 232 with external threads 234 sized to mate with internal threads 214 of sleeve 210. Plug 230 has a head 236 with a drive socket 238, preferably the same as drive socket 220 to reduce the number of tools required to insert anchor system 200.

Referring to FIG. 8, an alternative embodiment of a plug 330 is shown. Plug 330 is identical to plug 230 with a body 332 having external threads 334 and a head 336 with a drive socket 338, preferably the same as drive socket. External threads 334 of plug 330, however, can be broken to form a longitudinal channel 340. Although not shown, a diametrically opposite channel can also be formed. This channel and channel 340 form a passage for suture 100 to extend along plug 330 without interfering with plug 330.

Both plug 230 and plug 330 can be constructed from poly ether ether ketone (PEEK) or Polyethylene (PE) with a titanium spray.

To use system 200 with suture 100, sleeve 210 is inserted into the posterior hole drilled into the fibula. A non-lassoed end of suture 100 is inserted into sleeve 210 and threaded through a lasso loop (not shown) of a corresponding anterior tibial drill hole. Suture 100 is pulled tight so that the lasso goes into sleeve 210. Plug 230 is inserted into sleeve 210, pushing lassoed end of suture 100 into sleeve 210 and, with manual tightening of plug 230 in sleeve 210, system 200 captures and locks suture 100 in channel 222 in sleeve 210 and channel 340 is plug 330 is used.

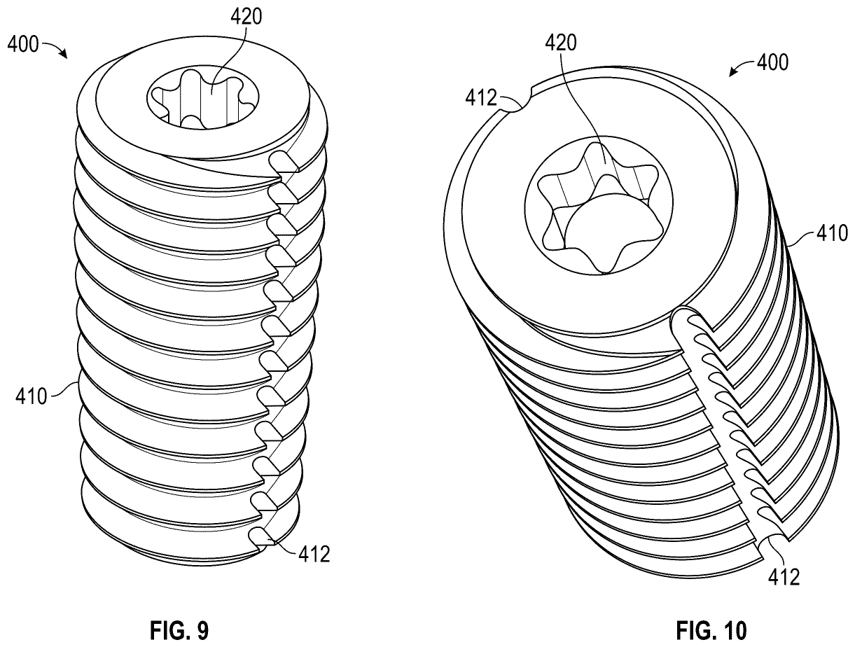

An alternative anchor system 400 is shown in FIGS. 9 and 10. Anchor system 400 can be a suture attached anchor. Anchor system 400 includes a generally cylindrical externally threaded body 410 having a pair of diametrically opposed exterior longitudinal channels 412 extending along an exterior thereof. Channels 412 each form a passage for suture 100 to extend along body 410 without interfering with body 410. Body 410 can be constructed from PEEK or PE or titanium with/without a titanium spray.

Anchor system 400 features a drive socket 420 formed therein. Drive socket 420 is sized to allow a driver, such as a Torx® driver (not shown), to engage drive socket 420 and rotate body 410 for insertion into an elbow, a hand, an ankle, a knee, a foot, or other appropriate body part.

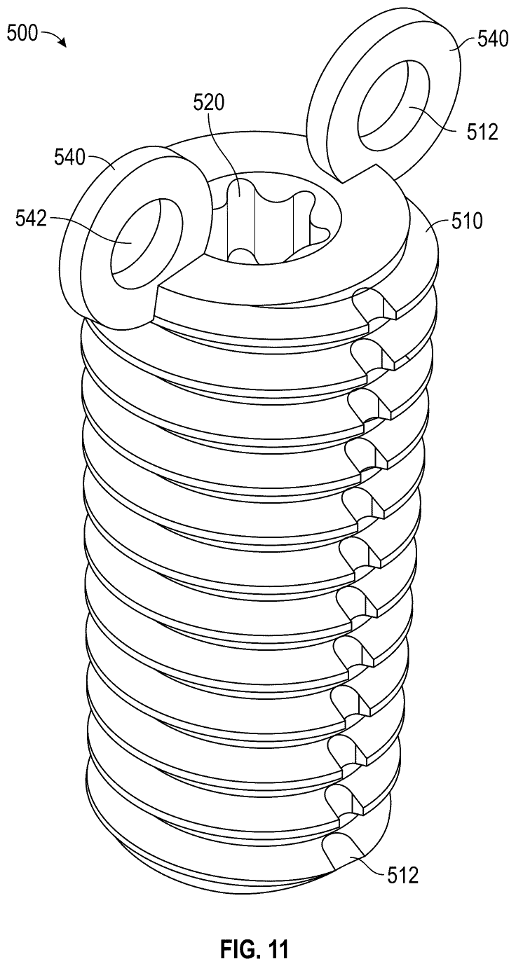

Still another alternative anchor system 500 is shown in FIG. 11. Anchor system 500 can be a suture receiving anchor. Anchor system 500 is similar to anchor system 400 with a generally cylindrical externally threaded body 510 having a pair of diametrically opposed exterior longitudinal channels 512 extending along an exterior thereof. Channels 512 each form a passage for suture 100 to extend along body 510 without interfering with body 510. Body 510 can be constructed from PEEK or PE or Titanium with/without a titanium spray

Anchor system 500 features a drive socket 520 formed therein. Drive socket 520 is sized to allow a driver, such as a Torx® driver (not shown), to engage drive socket 520 and rotate body 510 for insertion into an elbow, a hand, an ankle, a knee, a foot, or other appropriate body part.

Anchor system 500 differs from anchor system 400 by further having a pair of ears 540 having through openings 542 formed therein on diametrically opposing sies of drive socket 520 for passing suture 100 therethrough.

Anchor systems 400, 500 can be paired together and used for ligament reconstruction.

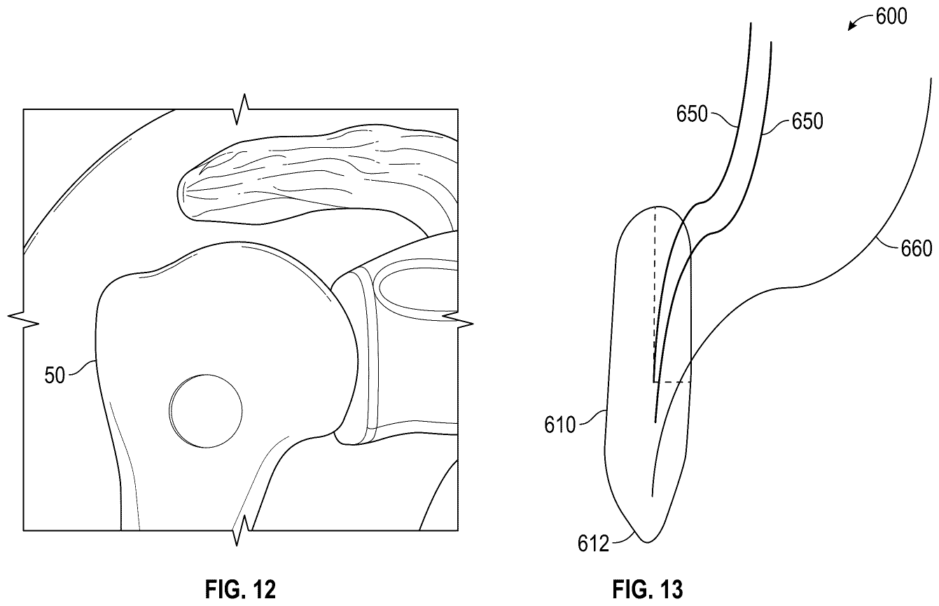

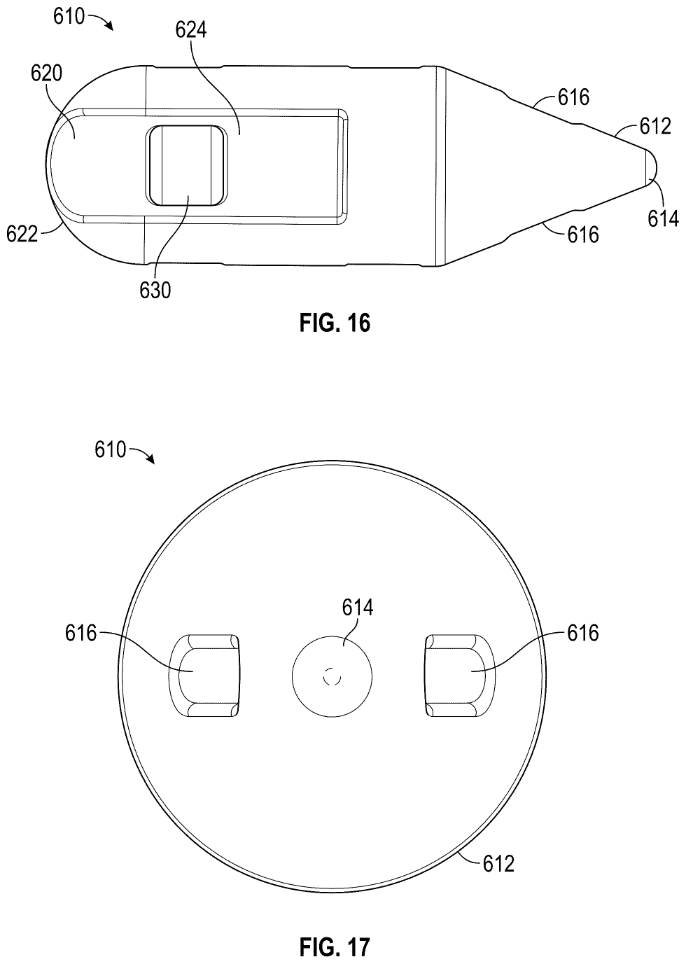

An alternative embodiment of an anchor system 600 according to the present invention is shown in FIGS. 12-21. Anchor system 600 can be used for rotator cuff repair, in an augmented or non-augmented fashion. Anchor system 600 can include a anchor 610 that can be inserted into the proximal humerus or other bone 50, shown in FIG. 12, with poor pull-out resistance.

Referring to FIG. 13, anchor 610 can be constructed from poly ether ether ketone (PEEK), titanium coated PEEK, titanium grade 4 rough surface, or other suitable biocompatible material. In an exemplary embodiment, anchor 610 can have a diameter between about 2.0 mm to about 5.5 mm., with the length of anchor 610 being about 3 to 4 times the diameter of anchor 610.

A tendon/ligament suture 650 can be located approximately half way the length of anchor, while a rotation suture 660 can be located toward a distal end 612 of anchor 610. Tendon/ligament suture 650 is used to secure anchor 610 into bone 50, while rotation suture 660 is used to rotate anchor 610 about 90 degrees after anchor 610 is inserted into bone, to secure anchor 610 inside bone 50 and prevent anchor 610 from pulling out of bone 50. Sutures 650, 660 can be the equivalent of suture 100 disclosed above, with the same features as suture 100.

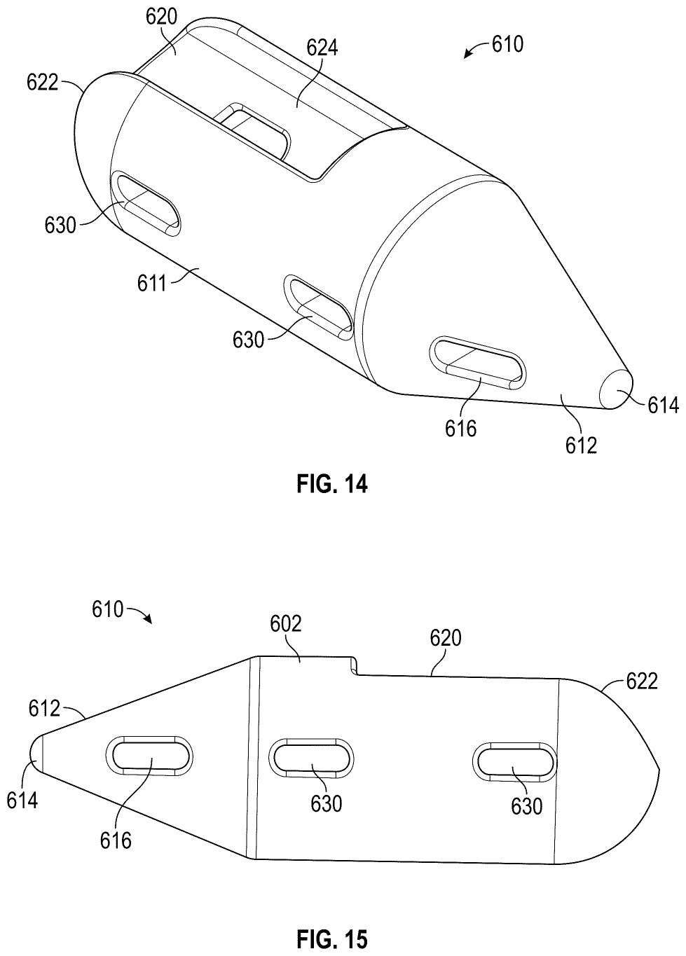

FIGS. 14-17 show an exemplary configuration for anchor 610. Anchor 610 includes a generally cylindrical body 611 with distal end 612 that terminates in a bullet tip 614 that is about ⅓ the overall length of anchor 610. A pair of diametrically opposing through-openings 616 are provided for rotation suture 660 to be inserted therethrough.

A top portion 620 of body 611 from an approximate center of body 611 toward proximal end 622 includes an open channel 624. Channel 624 has a length of about one half the overall length of anchor 610, with a width of between about 1 mm and about 3 mm, and a depth of about one half the diameter of anchor 610. Channel 624 is used to accommodate a delivery device 700, as will be described in detail below.

Body 611 includes a pair of generally oblong through openings 630 on either side of channel 624. Through openings 630 allow for the passage of tendon/ligament suture 650 through body 611. Another suture opening 632 in channel 624 is aligned with and extends into a proximal through opening 630.

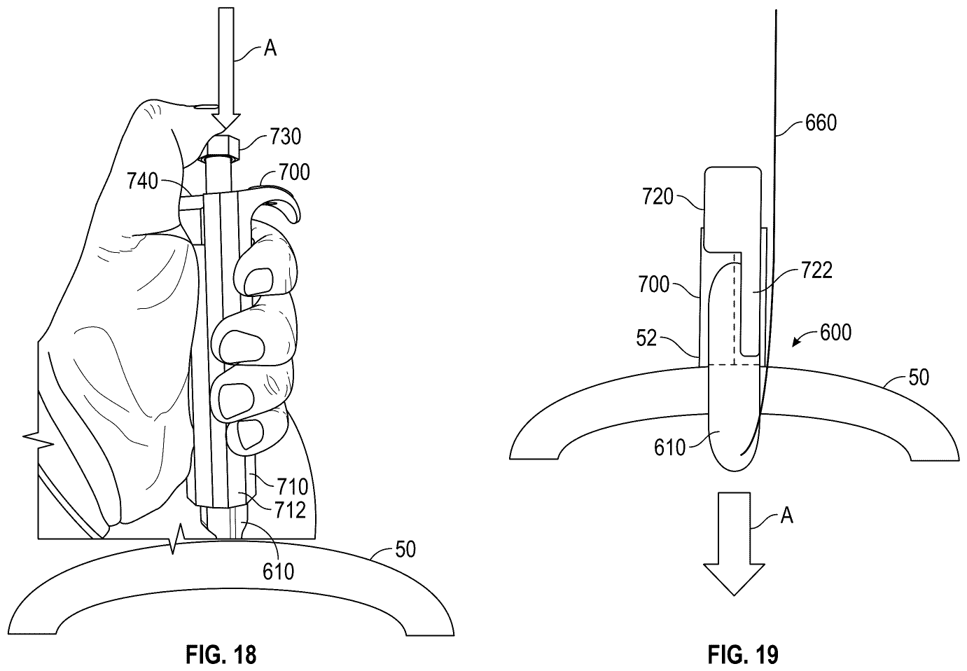

An anchor delivery device 700 (“device 700”), used to deliver anchor 610 into bone, is shown in FIGS. 18-21. Device 700 is similar to known devices used for arthroscopic surgery and is based on a modified calibrated pipette system. Device 700 uses a single handled delivery system as will be discussed below. Device 700 has a hollow tubular body 710 to allow anchor 600 to be inserted therein. Body 700 has an open distal tip 712 from which anchor 610 is discharged into an opening previously formed in bone 50.

As shown in FIG. 19, a plunger head 720 is slidingly disposed in body 710. Plunger head 720 includes a distal driver 722 that is sized to engage open channel 624 and drive anchor 610 distally into bone 50. Referring back to FIG. 18, a plunger 730 extends proximally from plunger head 720 and can be driven in the direction of arrow “A” in FIGS. 18 and 19 to drive anchor 610 and deliver anchor 610 into a pre-drilled hole 52 in bone 50. In an exemplary embodiment, diameter of hole 52 is about ⅓ the length of anchor 610

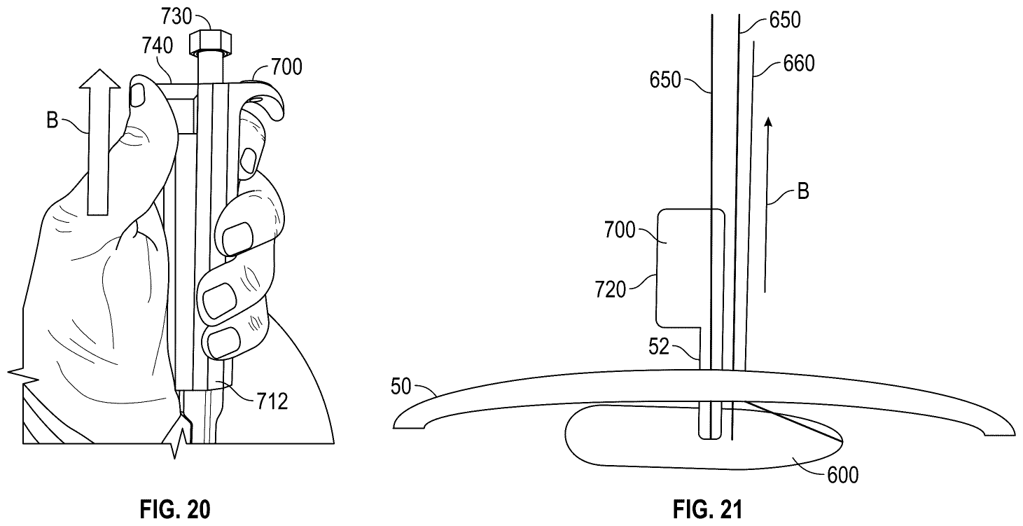

A rotation rod 740 is slidingly attached alongside body 710. Rotation suture 660 is attached to rotation rod 740 such that, when rotation rod 740 is slid proximally along arrow “B” in FIG. 20, rotation suture 660 is pulled proximally/superiorly, also in the direction of arrow “B”, shown in FIG. 21, to pivot anchor 610 about 90 degrees from the position shown in FIG. 19 to the position shown in FIG. 21. Sutures 650 act as a pivot for anchor 610 to pivot about when rotation suture 660 is pulled proximally/superiorly. When anchor 610 is rotated to the position shown in FIG. 21, anchor 610 cannot readily be pulled out of bone 50, providing a stable anchor for sutures 650 to be used to secure a tendon of ligament, as is required by the present procedure.

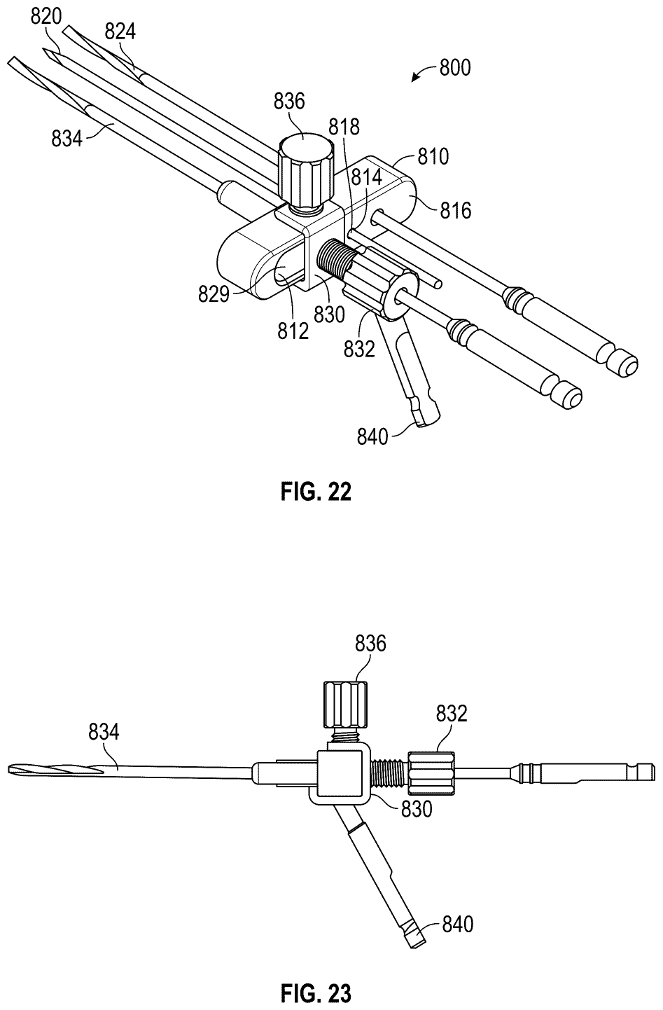

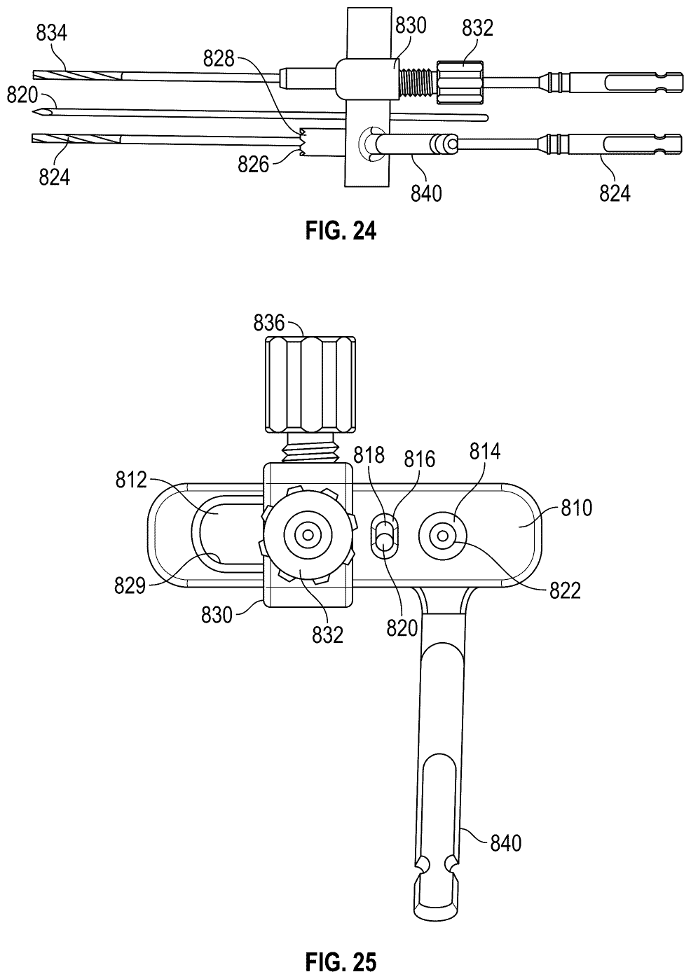

Referring to FIGS. 22-25, a syndesmosis jig assembly 800 (“assembly 800”) according to a first exemplary embodiment of the present invention is shown. Assembly 800 is used in the repair a syndesmosis injury by drilling through a fibula 50 and a tibia 60 whose joining ligaments have been ruptured, torn, or otherwise failed.

Assembly 800 includes a base member 810 having a tibia engaging portion 812, a central portion 814 adjacent to tibia engaging portion 812, and a fibula engaging portion 816 adjacent to central portion 814.

Central portion 814 includes a through-opening 818 sized to accept a K-wire 820. Fibula engaging portion 816 includes a through-opening 822 sized to allow a drill bit 824 to extend therethrough. Fibula engaging portion 816 includes a sleeve 826 extending distally from base member 810. Sleeve 826 includes a plurality of teeth 828 formed therein. Teeth 828 are used to bite into fibula 50 to prevent jig 800 from slipping while being used. Tibia engaging portion 812 includes a generally oblong shaped through-opening 829.

A slider 830 slides over base member 810 and aligns a tibia guide 832 with a desired location on tibia 60 to insert a drill bit 834 in tibia engaging portion 812. Tibia guide 834 can slide along oblong through-opening 829 to a desired location. A threaded lock 836 extends through slider 830 and can be screwed down onto base member 810 to secure slider 830 to base member 810.

A handle 840 extends downwardly at an oblique angle from base member 810 below fibula engaging portion 816. Handle 840 is used by a clinician to hold ad align jig 800.

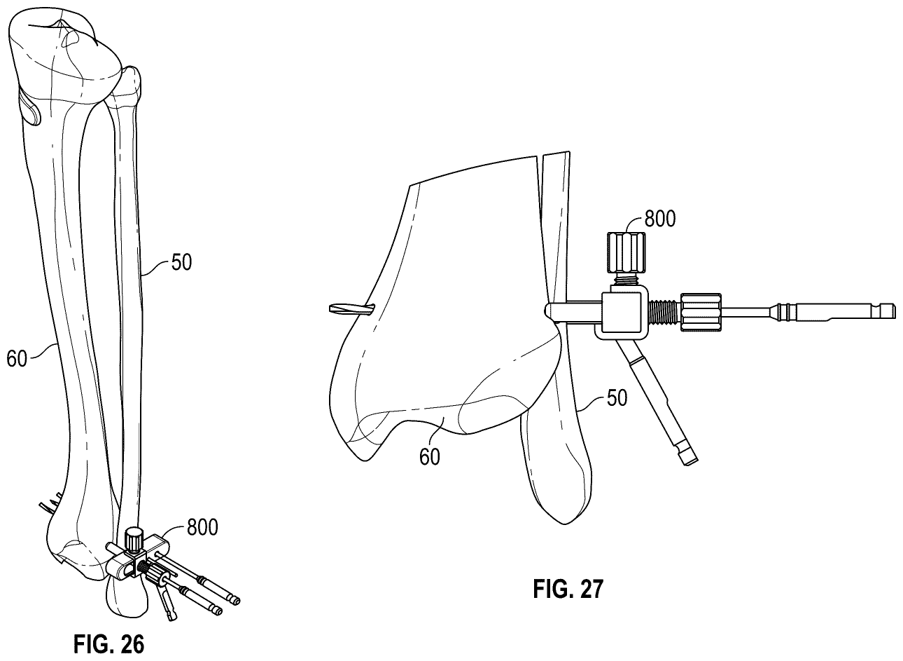

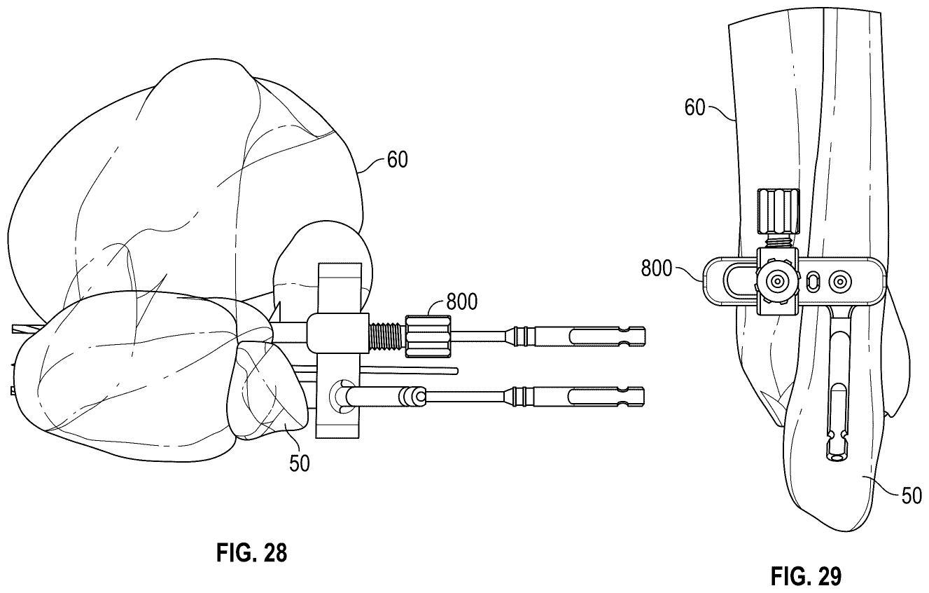

FIGS. 26-29 show an exemplary embodiment of a use of jig 800 with fibula 50 and tibia 60. After through-openings 52, 62, 64 are drilled through fibula 50 and tibia 60, respectively, jig 800 is removed.

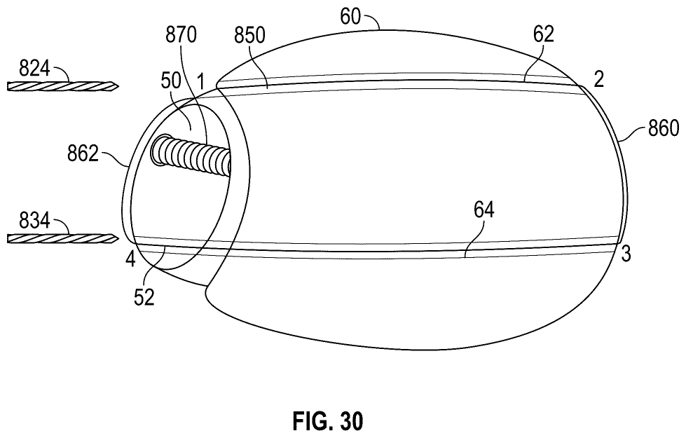

As shown in FIG. 30, a single suture 850 is used to secure fibula 50 to tibia 60 proximate to the foot (not shown) of patient. Suture 850 is inserted through through-opening 62 in tibia 60 and exits out of tibia 60. A first support strip 860 is placed against tibia 60 and suture 850 is advanced over first support strip 860 and into through-opening 64 and through-opening 52. Suture 850 is then wrapped around a screw 870, with a second support strip 862 on fibula 60. Screw 870 is screwed into the opening formed by K-wire 820, securing suture 850 to the fibula 50/tibia 60 without having to tie suture 850 into a knot.

In an exemplary embodiment, support strips 860, 862 can be constructed from poly ether ether ketone (PEEK), although those skilled in the art will recognize that other biocompatible materials can be used.

It will be further understood that various changes in the details, materials, and arrangements of the parts which have been described and illustrated in order to explain the nature of this invention may be made by those skilled in the art without departing from the scope of the invention as expressed in the following claims.

I claim:

1. A suture and anchor delivery system comprising:
a rotation suture;
a tendon/ligament suture;
an anchor comprising:
a generally cylindrical body having a length, a width, a proximal end, and a distal end terminating in a bullet tip, the bullet tip having a first pair of diametrically opposing through-openings, wherein the rotation suture is inserted therethrough, the body further having a second pair of diametrically opposed through openings, wherein the tendon/ligament suture is inserted therethrough;
and
an anchor delivery device comprising:
a hollow tubular body configured to allow the anchor to be inserted therein, the tubular body having an open distal tip from which the anchor is discharged; and
a plunger head slidingly disposed in the tubular body, the plunger head having a distal driver sized to engage the open channel and drive the anchor distally into a bone.

2. The suture and anchor delivery system according to claim 1, wherein the bullet tip comprises about ⅓ the length of the anchor.

3. The suture and anchor delivery system according to claim 1, wherein the anchor has length of the body is between 3 and 4 times the width of the body.

4. The suture and anchor delivery system according to claim 1, wherein the anchor is configured for implantation into a fibula.

5. The suture and anchor delivery system according to claim 1, wherein the tendon/ligament suture is located approximately half way the length of anchor.

6. The suture and anchor delivery system according to claim 1, wherein the body further comprises a top portion extending from an approximate center of the body toward the proximal end.

7. The suture and anchor delivery system according to claim 6, wherein the top portion comprises an open channel.

8. The suture and anchor delivery device according to claim 7, where in the channel is sized to accommodate the delivery device.

9. The suture and anchor delivery system according to claim 1, wherein the suture comprises a multifilament hybrid absorbable/non-absorbable braided surgical suture.

10. The suture and anchor delivery system according to claim 9, wherein the suture combines at least a first monofilament material and a second monofilament material, different from the first monofilament material.

11. The suture and anchor delivery system according to claim 10, wherein the first monofilament is bioabsorbable over a first period of time and wherein the second monofilament is bioabsorbable over a second period of time, longer than the first period of time.

12. The suture and anchor delivery system according to claim 11, wherein the first monofilament material consists of a material from the group selected from polyglycolic acid, polydioxanone, and combinations thereof.

13. The suture and anchor delivery system according to claim 10, wherein the first monofilament material is a bioabsorbable material.

14. The suture and anchor delivery system according to claim 13, wherein the second monofilament material is a non-bioabsorbable material.

15. The suture and anchor delivery system according to claim 14, wherein the second monofilament material is selected from the group consisting of nylon, silk, polyester, polyethylene, polypropylene, and combinations thereof.

16. The suture and anchor delivery system according to claim 14, wherein the second monofilament material comprises about 20% of the suture by weight.

17. The suture and anchor delivery system according to claim 1, further comprising a rotation rod slidingly attached alongside the body.

18. The suture and anchor delivery system according to claim 17, wherein the rotation suture is attached to the rotation rod such that, when the rotation rod is slid in a proximal direction, the rotation suture is pulled proximally/superiorly.

19. The suture and anchor delivery system according to claim 18, wherein, when the rotation suture is pulled proximally/superiorly, the anchor pivots about 90 degrees.

20. A syndesmosis jig assembly comprising:
a base member having:
a tibia engaging portion;
a central portion adjacent to the tibia engaging portion; and
a fibula engaging portion adjacent to the central portion.

21. The syndesmosis repair jig according to claim 20, wherein the central portion includes a through-opening sized to accept a K-wire.

22. The syndesmosis repair jig according to claim 20, wherein the fibula engaging portion includes a through-opening sized to allow a drill bit to extend therethrough.

23. The syndesmosis repair jig according to claim 20, wherein the fibula engaging portion includes a sleeve extending distally from the base member.

24. The syndesmosis repair jig according to claim 23, wherein the sleeve includes a plurality of teeth formed therein.

25. The syndesmosis repair jig according to claim 20, wherein the tibia engaging portion includes a generally oblong shaped through-opening.

26. The syndesmosis repair jig according to claim 20, further comprising a slider slidingly disposed over the base member and configured to align a tibia guide with a desired location on a tibia to insert a drill bit in the tibia engaging portion.

27. The syndesmosis repair jig according to claim 26, further comprising a threaded lock extending through the slider and configured to be screwed down onto the base member to secure the slider to the base member.

28. The syndesmosis repair jig according to claim 20, further comprising a handle extending downwardly at an oblique angle from the base membe below the fibula engaging portion.
